(19) United States
(12) Patent Application Publication
(10) Pub. No.: US 2018/0238796 A1
(43) Pub. Date: Aug. 23, 2018

(54) METHOD AND APPARATUS FOR STAIN TREATMENT

(71) Applicant: Conopco, Inc., d/b/a UNILEVER, Englewood Cliffs, NJ (US)

(72) Inventors: Deborah Jane COOKE, Chester (GB); Kenneth Stuart LEE, Higher Bebington, Wirral (GB)

(73) Assignee: Conopco, Inc., d/b/a UNILEVER, Englewood Cliffs, NJ (US)

(21) Appl. No.: 15/753,360

(22) PCT Filed: Aug. 19, 2016
(86) PCT No.: PCT/EP2016/069726

§ 371 Date: Feb. 19, 2018

(30) Foreign Application Priority Data
Aug. 24, 2015 (EP) 15182160.0

(51) Int. Cl. G01N 21/359; G01N 21/93; G01N 21/3563; G01N 21/95

(52) U.S. Cl. CPC G01N 21/359; G01N 21/93; G01N 21/3563; G01N 33/367; G01N 2201/0221; G01N 2201/129; G01N 21/95

(57) ABSTRACT

A method for identifying a stain on a textile (10), the method comprising the steps of: performing near-infrared reflectance spectroscopy to obtain spectral data from the stain over a range of wavelengths ranging from a shortest wavelength to a longest wavelength, the shortest wavelength having a value of 1595 nm or longer; accessing reference spectral data (33) of known stains; and comparing the spectral data of the stain to the reference spectral data of known stains to identify the stain.

### FIELD OF THE INVENTION

The present invention relates to methods and systems for identifying a stain on a textile, particularly to methods involving near-infrared reflectance spectroscopy.

### BACKGROUND OF THE INVENTION

There are many laundry products available for treating textiles, and the active agents within these products may vary depending on the specific textile product to be treated and also on the way in which treatment is to be applied. However, it is unusual for the average home user of laundry products to understand or appreciate the roles that each active agent will play and how best to remove specific stains.

As a result, home laundry is rarely targeted at the type of stain on a textile product. This “one-size-fits-all” approach can mean that the best cleaning results are not always achieved.

Specific treatment agents may be included in a product to have specific roles for the treatment of a particular type of stain. For example, enzymes work well for cleaning bodily fluids, food based greases and oils, milk-based stains, egg-based stains, grass. Within enzymes, particular enzymes can be chosen to optimise cleaning power for particular stains. For example: protease enzymes may be chosen to remove protein-based stains such as blood, egg, milk and grass; lipase enzymes may be more effective on oily or greasy stains such as butter, oil, gravy, cosmetics and lipstick; amylase enzymes remove starch-based stains such as gravy, potato, pasta, and rice. Bleaches are common in laundry products but a chlorine based bleach (e.g. sodium hypochlorite) can undesirably remove colour from clothes. An oxygen based bleach (e.g. sodium percarbonate or hydrogen peroxide) provides a safer alternative.

Other treatment agents may be present in a product such as sequestrants or builders, which may be included for various reasons, such as to assist the roles that surfactants play.

It may be desirable to perform steps separately, for example to have a pre-treating bleach step before using a more regular detergent.

In addition, different techniques may be used to administer the required treatment. Some cleaning products must be diluted before use, others will work best applied neat. Some cleaning products require time to work so cleaning cycles can beneficially involve soaking or steeping times.

The concentration of the treatment agent required may depend not only on the type of stain and type of fabric, but also on the seventy of the stain.

WO 2004/053220 discloses a method and apparatus for the identification of a parameter of the textile and discloses that near IR (NIR) spectroscopy at wavelengths of 369 to 1672 nm is particularly useful.

However, there is the need for an easy to use mechanism for identifying stains in the home by a consumer with a high success rate.

### SUMMARY OF THE INVENTION

Accordingly, the present invention aims to solve the above problems by providing, according to a first aspect, a method for identifying a stain on a textile, the method comprising the steps of: performing near-infrared reflectance spectroscopy to obtain spectral data from the stain over a range of wavelengths ranging from a shortest wavelength to a longest wavelength, the shortest wavelength having a value of 1595 nm or longer; accessing reference spectral data of known stains; and comparing the spectral data of the stain to the reference spectral data of known stains to identify the stain.

The range of wavelengths over which data is obtained is defined by: a lower bound corresponding to the shortest wavelength; and an upper bound corresponding to the longest wavelength. That is to say, the spectral data is obtained over the entire range (the whole range) of wavelengths including the shortest wavelength and the longest wavelength. The comparison of the spectral data of the stain to the reference spectral data is made over the entire range.

In this way, the near-infrared (NIR) spectroscopy captures a spectral fingerprint of the stain, providing mechanism for identifying stains which is non-destructive and easy to use. The method is therefore suitable for being performed by the owner of the textile item at home with no requirement for laboratory facilities.

By using a wavelength range which extends over longer wavelengths (i.e. starting a shortest wavelength of 1595 or longer), the NIR reflectance spectroscopy is less penetrating, i.e. more surface specific. This arises due to the fact that path length (penetration depth) decreases with increasing wavelength. An NIR reflectance spectrum taken over longer wavelengths will exhibit better defined bands and is therefore more informative as compared to similar NIR spectra taken at a range of lower wavelengths. It is also noted that light scattering increases with wavelength within the NIR spectrum. Overall, by utilising longer NIR wavelengths and in comparison to wavelengths used in the prior art, greater accuracy in predicting stains is obtained.

Optional features of the invention will now be set out. These are applicable singly or in any combination with any aspect of the invention.

The shortest wavelength may be 1600 nm. Alternatively, the shortest wavelength may be 1800 nm. Alternatively, the shortest wavelength may be 2000 nm.

According to a preferred embodiment, the range of wavelengths over which data is obtained is defined by: a lower bound corresponding to a shortest wavelength which is no shorter than 1600 nm; and an upper bound corresponding to a longest wavelength which is no longer than 2000 nm; and wherein the comparison of the spectral data of the stain to the reference spectral data is made over the entire range from 1600 nm to 2000 nm

The longest wavelength may be no longer than 2397 nm. Optionally, the longest wavelength may be no longer than 2200 nm. Optionally, it may be no longer than 2000 nm.

In one embodiment, the range of wavelengths over which reflectance is measured may extend from a shortest wavelength of 1595 nm to a longest wavelength of 2397 nm. In another embodiment, the range of wavelengths over which reflectance is measured may extend from a shortest wavelength of 2000 nm to a longest wavelength of 2397 nm. In an alternative embodiment, the range of wavelengths may extend from a shortest wavelength of 1800 nm to a longest wavelength of 2200 nm.

In one preferable embodiment, the range of wavelengths over which reflectance is measured may extend from a shortest wavelength which is no shorter than 1600 nm and a longest wavelength is no longer than 2000 nm.

Stains may be classified according to their composition (e.g. particulate, fatty), or their sensitivity to certain treatments (e.g. enzyme, bleach). Throughout this document, ‘stain type’ will refer to a combination of both of these designations (hence stain types include particulate, fatty, enzyme, and bleach).

By comparing the spectral data of the stain to known stains, it may be possible to match the spectral data to other stains of the same stain type (e.g. fatty, enzyme, bleach, and particulate). Preferably, we go one step further and match the spectral data from the stain to an actual stain identity e.g. Blood, Tomato, Sunflower Oil, Lipstick, Tangerine, Yellow Curry, Red Curry, Black Tea etc.

In addition to providing a fingerprint of the type of stain present, the spectral data taken from the NIR spectrometer may depend upon the type of textile being analysed. There may be a threshold thickness of stain at which the stain is too thick for the NIR signal to penetrate through to the textile itself. In such a scenario, the spectral data will not depend on the textile. The method could include a first step of deducing parameters of the textile itself, followed by measurements of the stain itself.

The method may further comprise the steps of: providing a user interface for the user treating the stain to provide information on the stain; and comparing the information provided at the user interface with information about the known stains.

The information obtained by the user may be used to filter the known stains before the spectral data from the stain is compared with known stains thereby reducing the number of comparisons that need to be made.

The user interface may be provided on a mobile device, wherein the mobile device could include: a mobile phone (cellphone), tablet, phablet, laptop and/or digital camera.

A mathematical pre-treatment applied to data such as first or second derivative. Known chemometric algorithms can be used to model the spectra and extract data.

The NIR absorption bands may be compared with known spectra stored in a library.

The user interface may be provided on a mobile device which includes a camera.

The user interface may be an application stored on the user's device or running remotely. It may be configured to interface with a camera on the user's device so that the method may include the step of taking a digital photograph of the stain using the mobile device.

The photograph taken by the digital camera may be processed to extract colour information about the stain to aid with identification. It may also be used to extract colour information about the textile itself. This could be particularly useful if the treatment is likely to include bleach. In this way a treatment which is non-damaging to the dyes of the coloured textile can be chosen. In some cases, the underlying colour of a textile may affect the colour of the stain. It is envisaged that colour information taken from an unstained part of the textile could also be used for calibration of the colour information retrieved by the camera to account for underlying textile colour and therefore determine the relative colour of the stain.

The photograph taken by the digital camera may also be used to determine background whiteness of a textile. By combining this background whiteness reading with the NIR spectrum of the white fabric enables the most appropriate treatment options to be chosen to improve background whiteness in addition to the stain removal.

Optionally, the user interface includes a typographical interface and/or drop down menu for a user to input information about the stain and/or the textile.

The input information may include the type of stain e.g. red wine, curry and may also include the type of fabric e.g. wool, cotton, and linen. This information provides further details that would be useful for determining how best to treat the stain.

In some embodiments, spectroscopy data can be sent from the NIR spectrometer to a mobile device. The mobile device could perform analysis of the spectroscopy data in combination with analysis of other data such as colour information in order to optimise the accuracy of the stain identification.

According to a second aspect of the present invention, there is provided a method of treating a textile comprising a stain, the method including the steps of: identifying a stain on the textile using the method of the first aspect; and choosing a treatment regime for treating the textile based on the identified stain.

According to a third aspect of the present invention, there is provided a stain determination system for identifying a stain on a textile including: a near-infrared reflectance (NIR) spectrometer which operates over a range of wavelengths, the range defined by a lower bound corresponding to a shortest wavelength; and an upper bound corresponding to a longest wavelength, the shortest wavelength being 1595 nm or longer; and an analysis module comprising a storage means, the analysis module configured to receive the spectral data from the NIR spectrometer; to retrieve reference spectral data of known stains from the storage means; and to compare the spectral data from the NIR spectrometer with the spectral data of known stains over the entire range of wavelengths, whereby a match between the NIR spectral data and the reference spectral data for a specific stain identifies the stain on the textile.

In a preferred embodiment, the range of wavelengths over which data is obtained is defined by: a lower bound corresponding to a shortest wavelength which is no shorter than 1600 nm; and an upper bound corresponding to a longest wavelength which is no longer than 2000 nm. Wherein the comparison of the spectral data of the stain to the reference spectral data is made over the entire range from 1600 nm to 2000 nm.

In this way, a non-destructive, easy to operate system is provided for analysing the stain and for outputting data that can be processed to determine how best to treat the stain. The system may provide further advantages e.g. low cost.

The NIR spectrometer is preferably handheld. By handheld, it should be understood that the NIR spectrometer forms a portable device contained in a handheld housing, the housing including a power source and NIR source such that no external connections to other devices such as lab bench equipment are required for a spectrum to be taken and for a stain to be identified.

Optionally, the stain determination system further comprises a treatment module, the treatment module configured to select treatment parameters based on the identified stain.

Optionally, the treatment module generates an output signal, the information in the signal being indicative of the identified stain. The output signal may be received by a treatment module and based on the information in that signal, appropriate treatment parameters can be chosen by the treatment module, the treatment parameters depending upon the identified stain.

Optionally, the analysis module includes a user interface configured to receive information about the stain from a user.

The user interface may take the form of an application located on a mobile device. It may be an application or website.

Further optional features of the invention are set out below.

### BRIEF DESCRIPTION OF THE DRAWINGS

Embodiments of the invention will now be described by way of example with reference to the accompanying drawings in which:

FIG. 1 shows an example of a method of treating a textile comprising a stain according to the present invention

FIG. 2 shows a schematic diagram of a stain determination system according to the present invention;

FIG. 3a shows an example of spectral data taken of a red curry stain; FIG. 3b shows a derivative plot of the spectral data of FIG. 3a;

FIG. 4a shows an example of spectral data taken of a yellow curry stain; FIG. 4b shows a derivative plot of the spectral data of FIG. 4a;

FIG. 5a shows an example of spectral data taken of a black tea stain; FIG. 5b shows a derivative plot of the spectral data of FIG. 5a;

FIG. 6a shows an example of spectral data taken of a black tea stain; FIG. 6b shows a derivative plot of the spectral data of FIG. 6a;

FIG. 7a shows the spectral data of FIGS. 3a and 4a on the same axes; FIG. 7b shows the derivative plots of FIGS. 3b and 4b on the same axes;

FIG. 8a shows the spectral data of FIGS. 5a and 6a on the same axes; FIG. 8b shows the derivative plots of FIGS. 5b and 6b on the same axes; and

FIG. 9a shows the spectral data taken from an Annatto oil stain and cooking oil stain on the same axes; FIG. 9b shoes the derivative plots of the spectral data of FIG. 9a.

### DETAILED DESCRIPTION AND FURTHER OPTIONAL FEATURES OF THE INVENTION

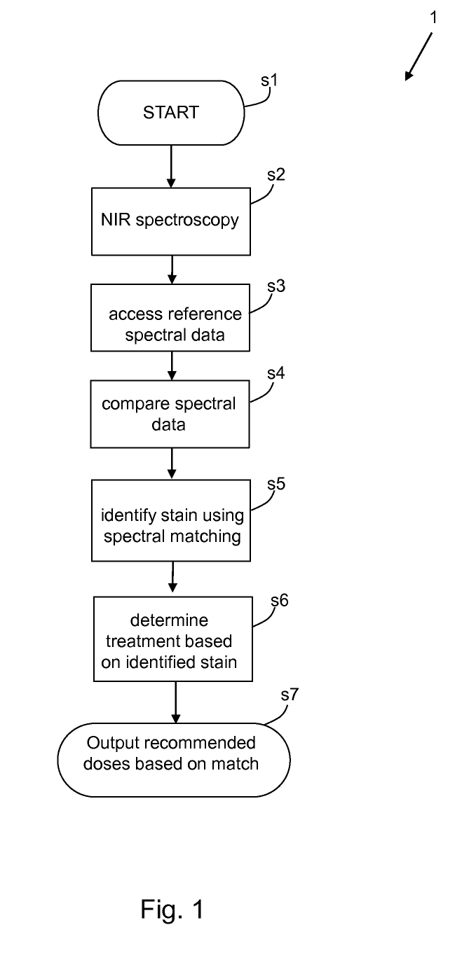

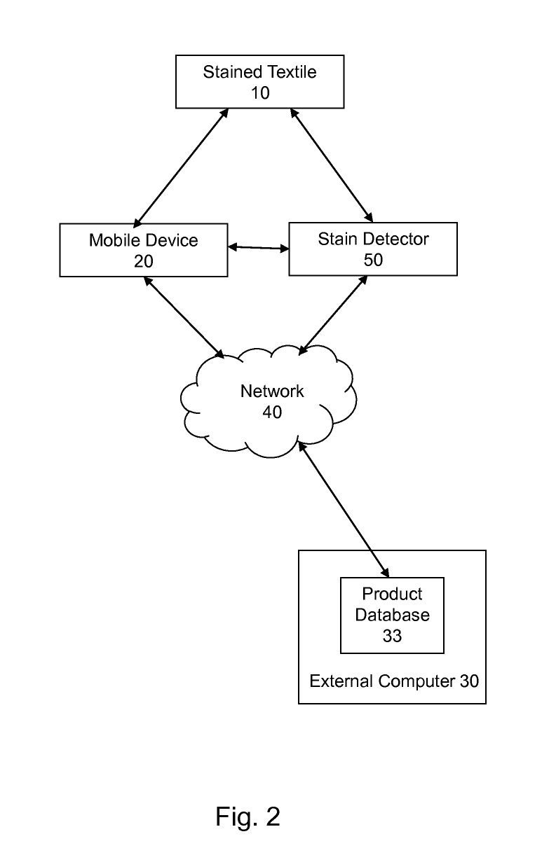

A method 1 and system for identifying a stain on a stained textile 10 are described below with reference to FIGS. 1 and 2.

A stain is identified s1 by an individual, who would typically be a typical consumer of commercial textile cleaning and treatment products.

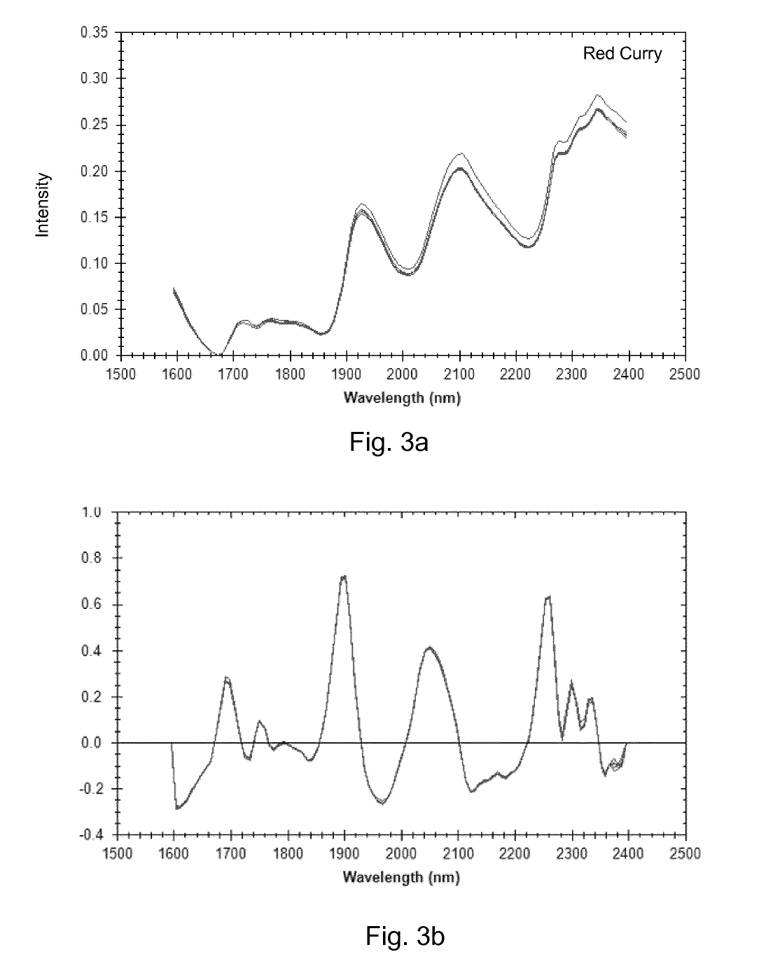

Using a stain detector 50 in the form of a near infra-red (NIR) spectrometer, an initial step, s2 of the method involves obtaining NIR spectral data from the stain. The NIR spectrometer is configured to illuminate the stain with radiation over a range of NIR wavelengths starting from a shortest wavelength up to a longest wavelength. The shortest wavelength is chosen to have a value of 1595 nm or longer in order to optimise the resulting spectral data. Reflectance signals from the stain are received by the Spectrometer and can be plotted to give a plot of reflected intensity vs wavelength. Examples of such spectra are shown in FIGS. 3-7.

Each set of measured spectral data obtained by the user via the NIR spectrometer is typically pre-processed before the step s4 of comparing the reference spectra to the user's measured spectral data.

This pre-processing of the user's measured spectral data may include the following pre-processing steps carried out using proprietary software (‘Method Generator’) from Thermo Scientific:
Each spectrum is processed to give its first derivative using the Savitzky-Golay (S. Golay) method with 3 smoothing points and degree=2 (i.e. second order of polynomial fit);
The derivative spectra are then normalised as follows: for each derivative spectrum the maximum (max) and minimum (min) intensity is found, each point in the spectrum is then scaled by 1/(max−min);

Reference spectral data for known stains are accessed, s3 by the user and are compared, s4 to the spectral data obtained by the NIR spectrometer. In some embodiments, the step of comparing the spectral data of the stain to reference spectral data of known stains includes performing multivariate data analysis techniques on the spectral data of the stain to be treated.

Reference spectral data could take the form of reference spectra obtained from repeated stains (e.g. five replicate stains) of each stain type on the textile of interest. The reference spectra may have been taken with a similar NIR instrument.

Each reference spectra is typically pre-processed before the step s4 of comparing the reference spectra to the user's measured spectral data.

This pre-processing of the reference spectral data may include the following pre-processing steps carried out using proprietary software (‘Method Generator’) from Thermo Scientific:
Each spectrum is processed to give its first derivative using the Savitzky-Golay method with 3 smoothing points and degree=2 (i.e. second order of polynomial fit);
The derivative spectra are then normalised as follows: for each derivative spectrum the maximum (max) and minimum (min) intensity is found, each point in the spectrum is then scaled by 1/(max−min);
This library of derivatised, smoothed and normalised reference spectra is then used for comparison s4 with the spectrum of the stain to be identified.

Spectral matching s5 compares the shape of each spectrum of the user's measured spectral data with each spectrum in the library (the user's spectral data having under gone the same pre-processing as the library spectra). The spectral matching step then assigns a “degree of match” value ranging from −1 (perfectly anti-matched) to +1 (perfect match) using a proprietary algorithm (Thermo Fisher). The library entries that have the highest match values to the unknown sample are then used to identify the unknown based on a voting scheme using k nearest neighbours (KNN).

For example, if an unknown stain sample was being compared to a library of stain spectra, and the number of neighbours is set to 3, and the three best matches were red curry, red curry, and yellow curry, then the unknown would be identified as red curry (2 out of 3)

Similarly, spectral matching can be applied just to the library itself (each spectrum in the library compared to every other spectrum in the library) to test how consistent/discriminating the library is.

Once a good quality library is obtained, an application is generated which can perform the required analysis. This application is loaded back onto the NIR instrument. Spectra of stains can then be acquired and analysed on the instrument, independent of the PC.

The instrument compares the sample spectrum with the library spectrum of the proposed identified stain. If the correlation threshold is greater than a specified value (the default being 0.95) then the instrument registers the identification. If this threshold isn't reached, then the sample is registered as unidentified. However, the best fit information can be obtained—i.e. the identity of the stain which gives the closest match.

Alternatively, once spectra have been transferred to a PC and saved as text files, the analysis as described above can be performed using a suitable software package such as MATLAB. In this case the pre-processed (derivatised, smoothed and normalised) spectra are subjected to discriminant analysis using a non-linear model (e.g. MATLAB fitcdiscr function).

The library/model can then be used to predict stain types using MATLAB predict function.

The stain detector comprising the NIR spectrometer is hand held and self-contained so is completely portable. In this way, whilst it can (as described above) be used in connection with PC analysis tools such as MATLAB, it is also capable of working in a stand-alone mode. In a stand-alone mode, there is no requirement whatsoever for the stain detector to be linked to external components via fibre optics or otherwise.

In some embodiments, the NIR spectrometer of the stain detector 50 may comprise no more than a single photodiode detector. The wavelengths from the incoming source radiation may be spatially separated by the diffraction grating across a MEMS (Micro-electrical Mechanical) chip. The desired wavelengths of light may be selected by the MEMS pixel masks. Light that is not diffracted is recombined at the grating and is measured by a single element photodiode detector. However, it is clear that this is only one example of a mechanism by which NIR spectroscopy at the required wavelengths could take place.

In addition to the NIR spectrometer, the stain detector 50 typically comprises an analysis module configured to receive spectral data from the NIR spectrometer (not shown). One or more of pre-processing of spectral data, comparisons of the spectral data, and identification by spectral matching may be carried out by this analysis module.

The system for identifying a stain on a textile may comprise further components, in addition to the stain detector 50 as shown in FIG. 2. For example, a mobile device 20 configured to take digital photographs such as a mobile phone (cellphone), tablet, phablet, laptop and/or digital camera.

The mobile device 20 and the stain detector 50 may communicate with each other directly, or may communicate over a network 40, both with each other and also with external resources such as a product database 33 or spectral database (not shown) on an external computer 30.

### EXPERIMENTAL RESULTS

### Example 1—Full Strength Stains

NIR spectral data were obtained for each of stains 1-24 applied to a cotton textile. The stains were given letter codes a-to-x respectively.

The NIR spectra were taken over a range of wavelengths ranging from a shortest wavelength of 1595 nm to a longest wavelength of 2397 nm.

TABLE 1
results for full strength stains

| | Stain | | |
|---|---|---|---|
| Stain | no. | Stain ID | Stain Type |
| Blood | 1 | a | Enzyme |
| TomatoSunflowerOil | 2 | b | Fatty |
| Lipstick | 3 | c | Fatty |
| Tangerine | 4 | d | Enzyme |
| Blank1 | 5 | e | t |
| ChocIceCreamPremium | 6 | f | Enzyme |
| BlackberryFruit | 7 | g | Bleach |
| ChocIceCreamEconomy | 8 | h | Enzyme |
| BlackShoePolish-CSS1 | 9 | i | Fatty |
| Gravy-Instant | 10 | j | Enzyme |
| GardenSoil(1:1water) | 11 | k | Particulate |
| RedSoil | 12 | l | Particulate |
| YellowPotteryClay | 13 | m | Particulate |
| ChocPudding | 14 | n | Enzyme |
| YellowCurry | 15 | o | Fatty |
| RedCurry | 16 | p | Fatty |
| MakeUp1 | 17 | q | Fatty |
| BlackCurrantJuice | 18 | r | Bleach |
| BlackTea | 19 | s | Bleach |
| RedWine | 20 | t | Bleach |
| LardVioletDye | 21 | u | Fatty |
| AnnattoOil | 22 | v | Fatty |
| CookingOilVioletDye | 23 | w | Fatty |
| Mascara | 24 | x | Fatty |

The resulting spectral data (i.e. the signatures acquired) were then compared with known values using the method described in more detail in the detailed description above.

During acquisition the spectra looked consistent, with the exceptions of e, k and x, for which it was found there may be some outliers. This comparison was by visual inspection of the baseline adjusted spectra, either on the instrument at the time of data collection, or on the PC via spectral matching, again as described in more detail above.

FIGS. 3 to 6 show the level of consistency within a stain (i.e. within a given stain identity such as a red curry stain). FIGS. 3a, 4a, 5a and 6a show the raw spectra processed with a ‘baseline off set’. FIGS. 3b, 4b, 5b and 6b show a derivative of the respective spectral data.

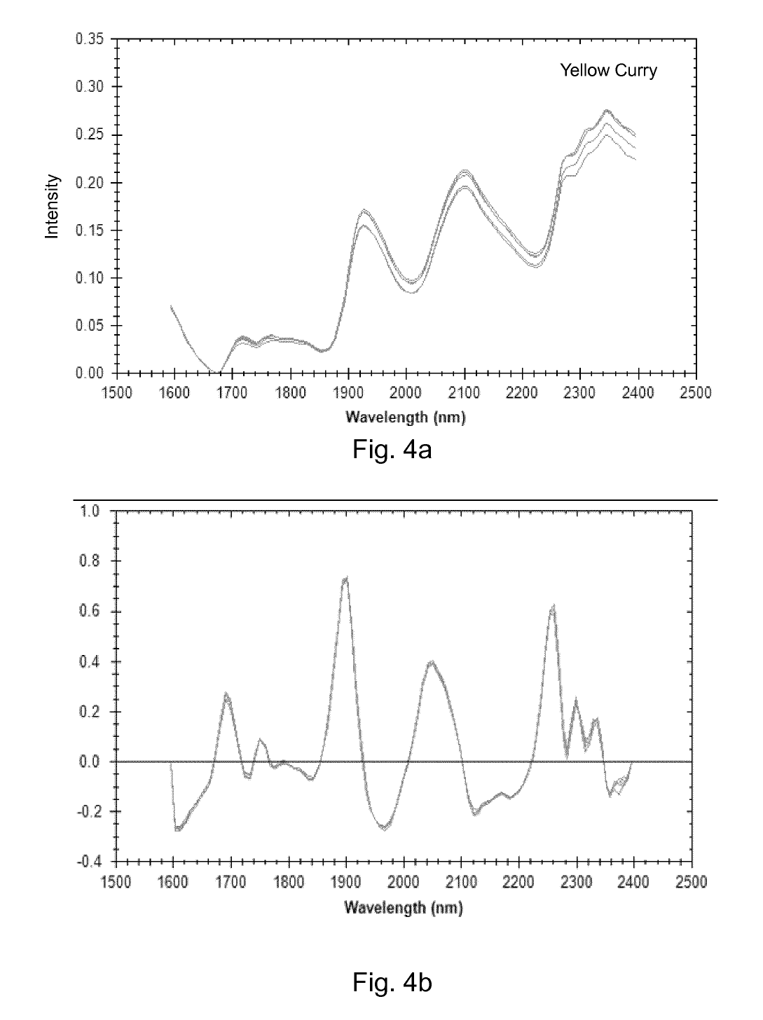

In more detail, FIG. 3a shows an example of spectral data taken of a red curry stain and FIG. 3b shows a derivative plot of the spectral data of FIG. 3a. FIG. 4a shows an example of spectral data taken of a yellow curry stain; FIG. 4b shows a derivative plot of the spectral data of FIG. 4a.

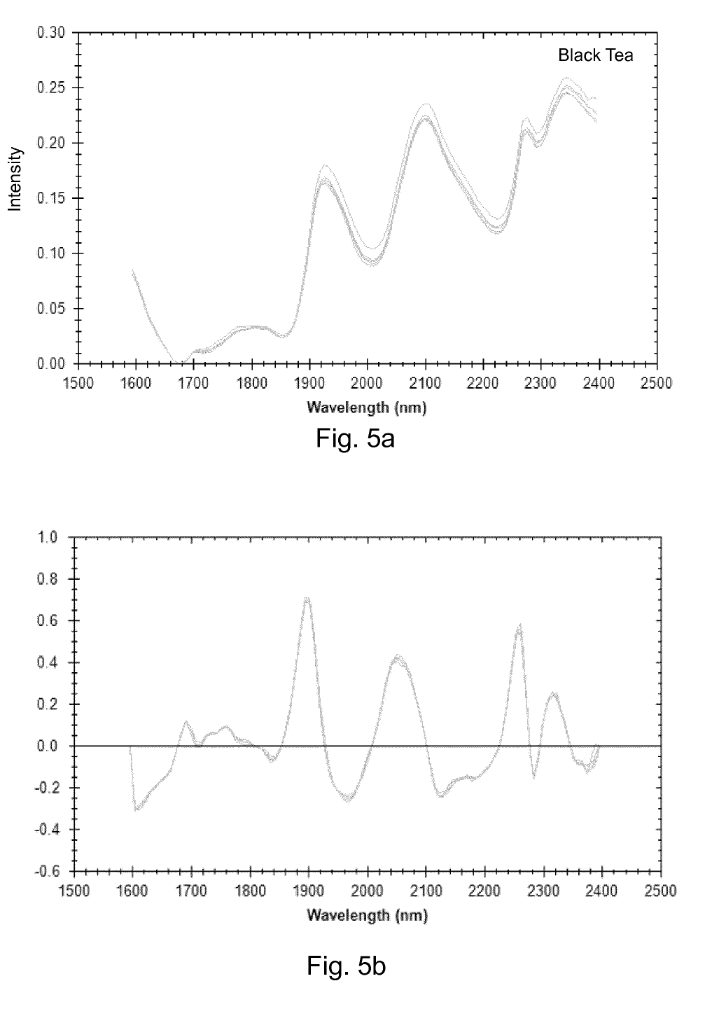

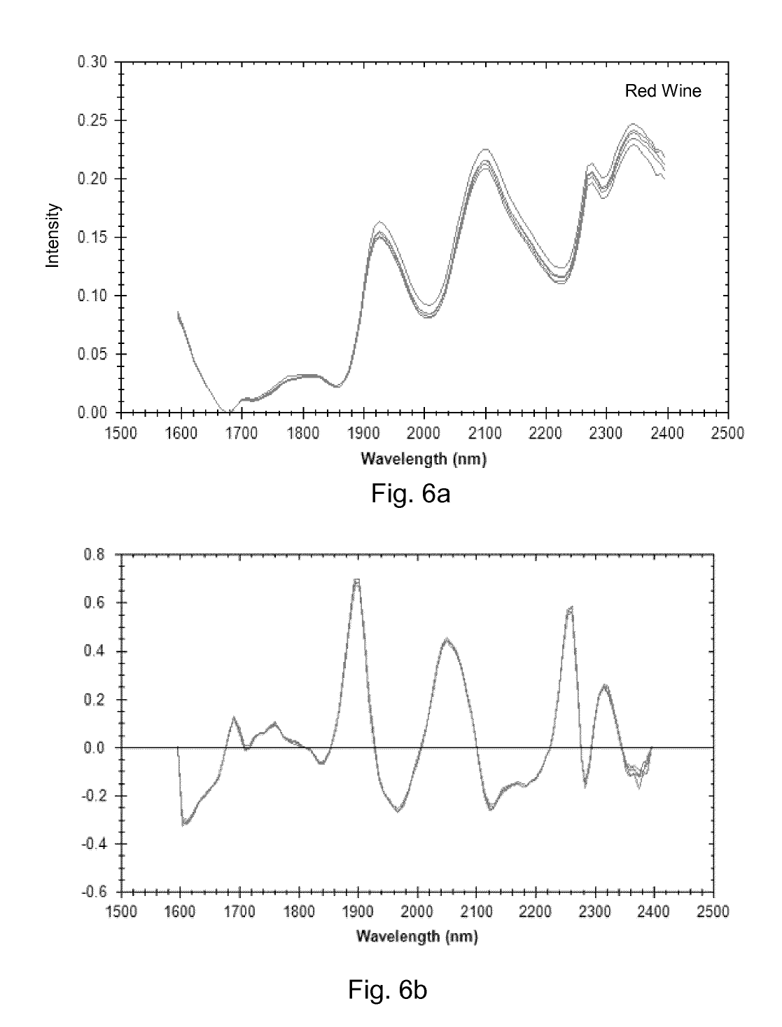

FIG. 5a shows an example of spectral data taken of a black tea stain; FIG. 5b shows a derivative plot of the spectral data of FIG. 5a. FIG. 6a shows an example of spectral data taken of a red wine stain; FIG. 6b shows a derivative plot of the spectral data of FIG. 6a.

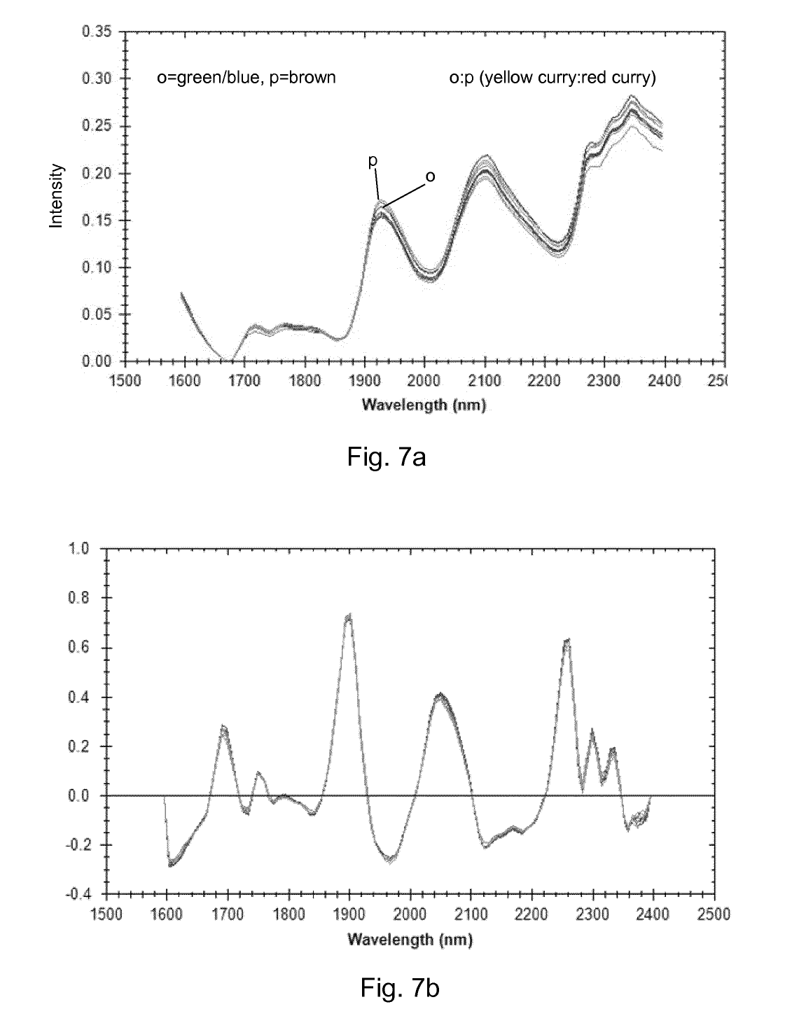

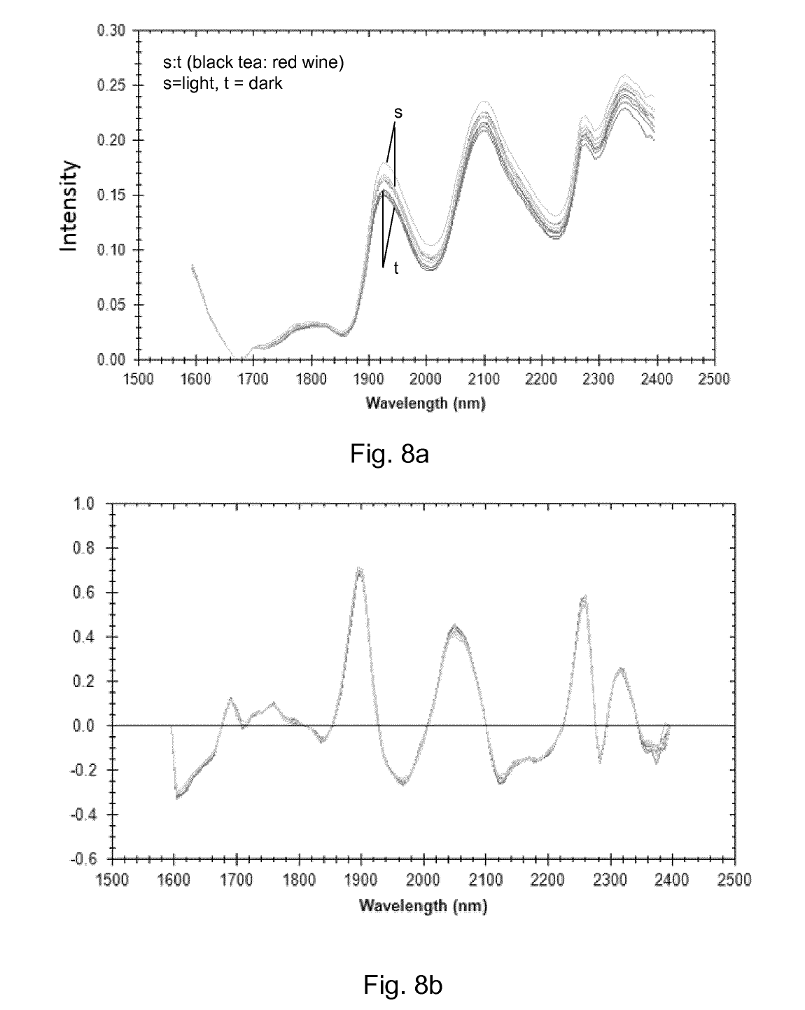

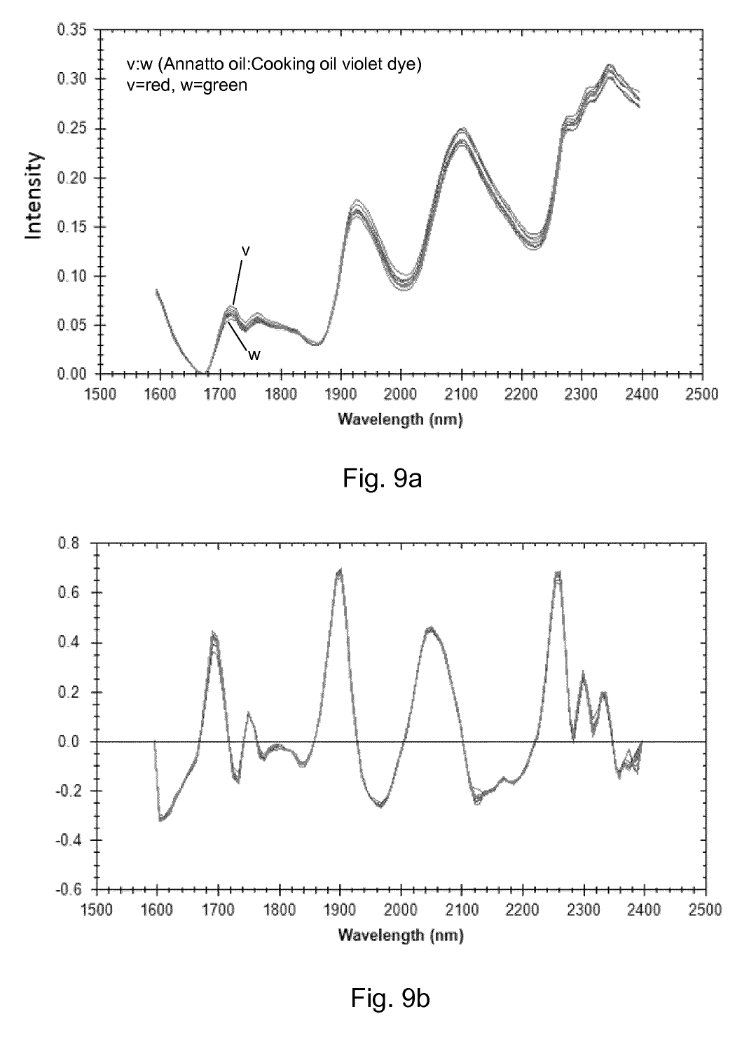

Mismatches between the identified stain and the actual stain are shown in Table 2 below as well as in FIGS. 7 to 9. FIG. 7 depicts the spectral data for stains “o” and “p” (yellow curry: red curry), FIG. 8 depicts the spectral data for stains “s” and “t” (black tea: red wine), and FIG. 9 depicts the spectral data for stains “v” and “w” (Annatto oil: Cooking oil violet dye).

It is noted that in all three cases, the mismatches are between stains within the same stain type (e.g. fatty) (including stain type sensitivity e.g. bleach sensitive) so the stain type itself (e.g. fatty or bleach) was correctly determined.

TABLE 2
mismatch of stain identification but not stain type sensitivity

| | No. of | |
|---|---|---|
| Preprocessing | mismatches | Mismatches |
| S. Golay (1 3 2) | 11 | o:p (yellow curry:red curry |
| Normalised Range | (90.75% correct) | (type = fatty)) |
| | | s:t (black tea:red wine (type = |
| | | bleach)) |
| | | v:w (Annatto oil:Cooking oil |
| | | violet dye (type = fatty)) |

FIG. 7a shows an example of the spectral data of FIGS. 3a and 4a on the same axes; FIG. 7b shows the derivative plots of FIGS. 3b and 4b on the same axes. FIG. 8a shows the spectral data of FIGS. 5a and 6a on the same axes; FIG. 8b shows the derivative plots of FIGS. 5b and 6b on the same axes; and FIG. 9a shows the spectral data taken from an Annatto oil stain and cooking oil stain on the same axes; FIG. 9b shows the derivative plots of the spectral data of FIG. 9a. From the spectral data shown it is clear that whilst the spectra were mismatched in terms of stain identity (i.e. by actual stain such as red curry or yellow curry), they are actually matched by stain type (e.g. fatty or bleach).

Table 2 shows that the library for fresh stains is self-consistent to better than 90% in terms of actual stain (i.e. 90.7% of stains were consistent in terms of type) and 100% self-consistent in terms if stain type.

### Example 2—Washed Stains

### Washed Stains

This example concerned stains on a test textile of cotton (the same stains mentioned above in relation to Example 1). As described above in relation to Example 1, NIR measurements were taken of the stains, but this time, the test textile was washed first using a commercial liquid. This Example therefore attempted to identify washed stains using the model built for fresh stains.

Again, the method of the present invention was then carried out using the stain detection system 50 to identify the washed stains.

TABLE 3
Results for washed stains

| | Stain | | Stain |
|---|---|---|---|
| Stain | no. | Stain ID | Assignment |
| Blood | 1 | a | t |
| Tomato Sunflower Oil | 2 | b | t |
| Lipstick | 3 | c | p |
| Tangerine | 4 | d | t |
| Blank1 | 5 | e | t |
| Choc Ice Cream | 6 | f | t |
| Premium | | | |
| Blackberry Fruit | 7 | g | s |
| Choc Ice Cream | 8 | h | t |
| Economy | | | |
| Black Shoe Polish | 9 | i | i |
| Gravy-Instant | 10 | j | s |
| Garden Soil(1:1 water) | 11 | k | e |
| Red Soil | 12 | l | s |
| Yellow Pottery Clay | 13 | m | t |
| Choc Pudding | 14 | n | t |
| Yellow Curry | 15 | o | s |
| Red Curry | 16 | p | t |
| Make Up1 | 17 | q | t |
| Black Currant Juice | 18 | r | t |
| Black Tea | 19 | s | t |
| Red Wine | 20 | t | s |
| Lard Violet Dye | 21 | u | p |
| Annatto Oil | 22 | v | t |
| Cooking Oil Violet Dye | 23 | w | t |
| Mascara | 24 | x | k |

As can be seen from Table 3 above, the spectral matching model (which was built for fresh stains) failed to identify washed stains.

A new model was then built based on the spectra acquired from the washed stains. This model was then tested for internal consistency. Mismatches in stain type are shown in bold font in Table 4 below:

TABLE 4
mismatches in stain type sensitivity for washed stains

| Letter Codes | Number Codes | Stain Type |
|---|---|---|
| a h | 1 8 | Enz. Enz |
| a d | 1 4 | Enz. Enz. |
| e h | 5 8 | Redep Enz. (both of |
| | | these there's very |
| | | little visual stain) |
| e a | 5 1 | Redep Enz. |
| e n | 5 14 | Redep Enz. (both of |
| | | these there's very |
| | | little visual stain) |
| g d | 7 4 | Bleach Enz. |
| g e | 7 5 | Bleach Redep |
| h e | 8 5 | Enz. Redep (both of |
| | | these there's very |
| | | little visual stain) |
| h n | 8 14 | Enz. Enz. |
| l t | 12 20 | Part. Bleach |
| m s | 13 19 | Part. Bleach |
| m l | 13 12 | Part. Part. |
| m h | 13 8 | Part. Enz. |
| o p | 15 16 | Fatty Fatty |
| q w | 17 23 | Fatty Fatty |
| r t | 18 20 | Bleach Bleach |
| r s | 18 19 | Bleach Bleach |
| s m | 19 13 | Bleach Part. |
| s r | 19 18 | Bleach Bleach |
| s t | 19 20 | Bleach Bleach |
| t s | 20 19 | Bleach Bleach |
| t r (+duplicates) | 20 18 | Bleach Bleach |
| v w (+duplicates) | 22 23 | Fatty Fatty |

From the results of Table 4, it can be seen that only ten (shown in bold above in the Stain Type column) out of 120 measurements are the wrong stain type.

Combining data from the washed stains and fresh stains gave a high level of consistency within the resulting library of stains. The spectral match accuracy within the library (i.e. correct stain identities) is 85%. The actual level of miss-matches in terms of stain type across the whole set is ˜3.5%

### Example 3—Analysis by Stain Type

In this example, the combined models (corresponding to both fresh stain and washed stain libraries) were combined and the application was then used on the instrument on textile samples.

As with Examples 1 and 2 above, NIR spectral data were obtained for each of stains 1-24 applied to a cotton textile. The stains were given letter codes a-to-x respectively.

The NIR spectra were taken over a range of wavelengths ranging from a shortest wavelength of 1595 nm to a longest wavelength of 2397 nm.

In the table below the results columns indicate the stain type by their initials:

Enzyme=E

Fatty=F

Particulate=P

Bleach=B

Note that there were two measurements taken (two separate stains of each stain type) so the table below includes two results for each sample.

TABLE 5
comparison of results on concentrated and washed samples

| | | | | Result on | |
|---|---|---|---|---|---|
| | | | | concen- | Result |
| | Stain | Stain | Stain | trated | on washed |
| Stain | no. | ID | Type | sample | sample |
| Blood | 1 | a | Enzyme | EE | PE |
| TomatoSunflowerOil | 2 | b | Fatty | FF | PE |
| Lipstick | 3 | c | Fatty | FF | FF |
| Tangerine | 4 | d | Enzyme | EE | EE |
| Blank1 | 5 | e | Redep | RR | PE |
| ChocIceCreamPremium | 6 | f | Enzyme | EE | PE |
| BlackberryFruit | 7 | g | Bleach | BB | EE |
| ChocIceCreamEconomy | 8 | h | Enzyme | EE | EE |
| BlackShoePolish | 9 | i | Fatty | FF | FF |
| Gravy-Instant | 10 | j | Enzyme | EE | EE |
| GardenSoil(1:1water) | 11 | k | Partic- | PP | PP |
| | | | ulate | | |
| RedSoil | 12 | l | Partic- | PP | PE |
| | | | ulate | | |
| YellowPotteryClay | 13 | m | Partic- | PP | PE |
| | | | ulate | | |
| ChocPudding | 14 | n | Enzyme | EE | EE |
| YellowCurry | 15 | o | Fatty | FF | FF |
| RedCurry | 16 | p | Fatty | FF | EF |
| MakeUp1 | 17 | q | Fatty | FF | FP |
| BlackCurrantJuice | 18 | r | Bleach | BB | EE |
| BlackTea | 19 | s | Bleach | BB | BE |
| RedWine | 20 | t | Bleach | RR | PE |
| LardVioletDye | 21 | u | Fatty | FF | FF |
| AnnattoOil | 22 | v | Fatty | FF | FF |
| CookingOilVioletDye | 23 | w | Fatty | FF | FF |
| Mascara | 24 | x | Fatty | RU | FF |
| | | | | 8% | 34% |
| | | | | wrong | wrong |
| | | | | 92% | 66% |
| | | | | correct | correct |

Table 5 shows the results obtained by using the combined model, loading this onto the instrument and then using this application to identify new stains on textile samples. This contrasts with Tables 2 and 4 which show the results of self-consistency testing within the libraries.

Combining data from the washed stains and fresh stains (as described at the end of Example 2) gave a library for which the spectral match accuracy (i.e. correct stain identities) was 85%. The actual level of miss matches in terms of stain type across the whole set was ˜3.5%

Table 5 shows that when the combined library (consisting of spectra of fresh stains and washed stains) is compiled into an application and used in stand-alone mode on the NIR instrument it is 92% successful in identifying unwashed stains on fabric, but only 66% accurate in identifying washed stains on fabric.

### Example 4—Stains Obtained at Full Concentration

Here, the original stain library from Example 1 was put it onto the stain detector 50 and this was used to identify actual stains on fabric at full strength.

TABLE 6
Full Strength

| Stain no. | Stain ID | Stain Type | Result on conc sample | | | |
|---|---|---|---|---|---|---|
| 1 | a | Enzyme | a | a | a | a |
| 2 | b | Fatty | b | b | b | b |
| 3 | c | Fatty | c | c | c | c |
| 4 | d | Enzyme | d | d | d | d |
| 5 | e | Blank | e | e | e | e |
| 6 | f | Enzyme | f | f | f | f |
| 7 | g | Bleach | g | g | g | g |
| 8 | h | Enzyme | h | h | h | h |
| 9 | i | Fatty | i | i | i | i |
| 10 | j | Enzyme | j | j | j | j |
| 11 | k | Particulate | k | k | k | k |
| 12 | l | Particulate | l | l | l | l |
| 13 | m | Particulate | m | m | m | m |
| 14 | n | Enzyme | n | n | n | n |
| 15 | o | Fatty | p | b | p | o |
| 16 | p | Fatty | p | p | p | p |
| 17 | q | Fatty | q | q | q | q |
| 18 | r | Bleach | r | d | d | r |
| 19 | s | Bleach | e | e | r | t |
| 20 | t | Bleach | e | r | r | r |
| 21 | u | Fatty | u | u | u | u |
| 22 | v | Fatty | w | w | w | w |
| 23 | w | Fatty | w | w | w | w |
| 24 | x | Fatty | (x) | (x) | (i) | (x) |

Out of 86 assignments shown in Table 5:

5 are wrong type (bold and italic)

9 are wrong assignment, but correct type (bold)

4 are unidentified, but for three of these the best fit is the correct stain, and for all four the best fit is the correct type (best fit shown in brackets).

During analysis, the instrument compares the sample spectrum with the library spectrum of the proposed identified stain. If the correlation threshold is greater than a specified value (e.g. 0.95) then the instrument registers the identification. If this threshold is not reached, then the sample is registered as unidentified. However, the best fit information can then be obtained to deduce the identity of the stain which gives the closest match.

Thus, the results show that:

The assignment of stain types is 100*(84−9)/84˜90% accurate

The assignment of actual stain is 100*(84−9−4)/84˜85%

### Example 5—Stains Obtained at Half and Quarter Strength

Where it was possible, NIR measurements of half and/or quarter strength were taken. These are summarised in Table 7 and 8 below. Only some stains were available at the required levels, so the table indicates only the stains that were available at these levels.

TABLE 6
Half Strength

| | | | |
|---|---|---|---|
| Stain | Stain | | Results on Half Strength |
| no. | ID | Stain Type | Samples |

| | | | | | |
|---|---|---|---|---|---|
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | h | h | f |
| 7 | g | Bleach | | | |
| 8 | h | Enzyme | r | d | h |
| 9 | i | Fatty | | | |
| 10 | j | Enzyme | e | j | j |
| 11 | k | Particulate | | | |
| 12 | l | Particulate | | | |
| 13 | m | Particulate | e | e | e |
| 14 | n | Enzyme | h | h | h |
| 15 | o | Fatty | e | e | e |
| 16 | p | Fatty | | | |
| 17 | q | Fatty | | | |
| 18 | r | Bleach | r | r | r |
| 19 | s | Bleach | e | e | e |
| 20 | t | Bleach | r | e | e |
| 21 | u | Fatty | | | |
| 22 | v | Fatty | | | |
| 23 | w | Fatty | | | |
| 24 | x | Fatty | | | |

8/27=correct stain identity (30%)

14/27=correct stain type sensitivity (52%)

TABLE 7
Quarter Strength

| | | | |
|---|---|---|---|
| Stain | Stain | | Results on Quarter Strength |
| no. | ID | Stain Type | Samples |

| | | | | | |
|---|---|---|---|---|---|
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | h | h | h |
| 7 | g | Bleach | | | |
| 8 | h | Enzyme | d | d | d |
| 9 | i | Fatty | | | |
| 10 | j | Enzyme | r | e | i |
| 11 | k | Particulate | | | |
| 12 | l | Particulate | | | |
| 13 | m | Particulate | e | e | e |
| 14 | n | Enzyme | d | b | d |
| 15 | o | Fatty | e | e | e |
| 16 | p | Fatty | | | |
| 17 | q | Fatty | | | |
| 18 | r | Bleach | e | e | e |
| 19 | s | Bleach | e | e | e |
| 20 | t | Bleach | e | e | e |
| 21 | u | Fatty | | | |
| 22 | v | Fatty | | | |
| 23 | w | Fatty | | | |
| 24 | x | Fatty | | | |

For quarter strength stains, only 8 out of 27 were assigned the correct stain type.

From these results, it can be seen that the library based on full strength stains is not applicable to the lower level stains. Libraries based on the spectral data taken from lower level stains have therefore been produced.

In one example, such a library is created from signatures acquired from stains at a 50% level (“half level stains”). In another example, a library is created from signatures acquired from stains at a 25% level (“quarter level stains”).

The paragraphs below describe the parameters used to build libraries based on the samples with lower levels of stains along with resulting success rates. These libraries were tested for internal consistency and shown to give a high degree of stain identification success (to ˜80%).

### For Half Level Stains Only

Golay (1,3,2) applied
Normalise Range

Gives 74% correct stain, 82% to correct stain type

No pattern to the remaining errors.

### For Quarter Level Stains

Golay (1,3,2) applied
Normalise Range

Gives 72% correct stain, 84% to correct stain type

No pattern to the remaining errors.

The half and quarter libraries were then combined to form a combined library (the “Lower Level Stains” library) with all of the data at both levels.

### For “Lower Level Stains”

Golay (1,3,2) applied
Normalise Range

Gives 68% correct stain, 79% to correct stain type

No pattern to the remaining errors

Finally, the Lower Level Stains library was combined with First Stains on Knitted Cotton to give “Stains_on_Cotton_All_Levels”.

### For “Stains on Cotton all Levels”

Golay (1,3,2) applied
Normalise Range

Gives 79% correct stain, 86% to correct stain type

Again, it is noted that a potential downfall of this analysis is that the resulting library includes some stains for which data are only present at the high level.

To address this concern, a model was built using just spectral data for which there were available stains at all 3 levels (quarter, half and full) resulting in “Stains on Cotton Three Levels”.

### For “Stains on Cotton Three Levels”

Golay (1,3,2) applied
Normalise Range

Gives 75% correct stain, 85% to correct stain type

This model was subsequently used to build an application, which was transferred to the NIR spectrometer and tested out on fabric test cloths prepared at the different stain levels, the results of which are summarised in Tables 8, 9, 10 and 11 below.

TABLE 8

| | | | |
|---|---|---|---|
| Stain | Stain | | Results on Full Strength |
| no. | ID | Stain Type | Samples |

| | | | | | |
|---|---|---|---|---|---|
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | f | f | f |
| 7 | g | Bleach | | | |
| 8 | h | Enzyme | h | n | n |
| 9 | i | Fatty | | | |
| 10 | j | Enzyme | j | j | j |
| 11 | k | Particulate | | | |
| 12 | l | Particulate | | | |
| 13 | m | Particulate | m | m | m |
| 14 | n | Enzyme | n | n | n |
| 15 | o | Fatty | o | o | o |
| 16 | p | Fatty | | | |
| 17 | q | Fatty | | | |
| 18 | r | Bleach | t | t | t |
| 19 | s | Bleach | m | s | m |
| 20 | t | Bleach | r | t | r |
| 21 | u | Fatty | | | |
| 22 | v | Fatty | | | |
| 23 | w | Fatty | | | |
| 24 | x | Fatty | | | |

Correct=18/27 (67%)

Correct type=25/27 (93%)

From these results, it is apparent that the application performs well when carried out on stains at full strength, where it yields a high percentage of correct stain identity and an extremely high percentage of correct stain type.

TABLE 9
results on half strength samples

| | | | |
|---|---|---|---|
| Stain | Stain | | Results on Half Strength |
| no. | ID | Stain Type | Samples |

| | | | | | |
|---|---|---|---|---|---|
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | o | o | f |
| 7 | g | Bleach | | | |
| 8 | h | Enzyme | h | h | h |
| 9 | i | Fatty | | | |
| 10 | j | Enzyme | o | j | j |
| 11 | k | Particulate | | | |
| 12 | l | Particulate | | | |
| 13 | m | Particulate | m | m | o |
| 14 | n | Enzyme | n | n | n |
| 15 | o | Fatty | o | r | o |
| 16 | p | Fatty | | | |
| 17 | q | Fatty | | | |
| 18 | r | Bleach | t | r | o |
| 19 | s | Bleach | t | m | m |
| 20 | t | Bleach | m | o | m |
| 21 | u | Fatty | | | |
| 22 | v | Fatty | | | |
| 23 | w | Fatty | | | |
| 24 | x | Fatty | | | |

Correct=13/27 (48%)

Correct type=16/27 (59%)

So, the application is less good on stains at half strength.

TABLE 10
results on quarter strength samples

| Stain no. | Stain ID | Stain Type | Results on Quarter Strength Samples | | |
|---|---|---|---|---|---|
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | f | o | f |
| 7 | g | Bleach | | | |
| 8 | h | Enzyme | h | h | h |
| 9 | i | Fatty | | | |
| 10 | j | Enzyme | j | j | j |
| 11 | k | Particulate | | | |
| 12 | l | Particulate | | | |
| 13 | m | Particulate | o | m | m |
| 14 | n | Enzyme | n | n | n |
| 15 | o | Fatty | o | m | s |
| 16 | p | Fatty | | | |
| 17 | q | Fatty | | | |
| 18 | r | Bleach | o | r | t |
| 19 | s | Bleach | s | blank | blank |
| 20 | t | Bleach | s | t | t |
| 21 | u | Fatty | | | |
| 22 | v | Fatty | | | |
| 23 | w | Fatty | | | |
| 24 | x | Fatty | | | |

Correct=18/27 (67%)

Correct type=20/27 (74%)

TABLE 11
comparison of results for three stain level model across all levels

| | % Correct stain | |
|---|---|---|
| Level/% | identity | % Correct Type |
| 100 | 67 | 93 |
| 50 | 48 | 59 |
| 25 | 67 | 74 |
| Total | 61 | 75 |

So, the new model (based on three stain levels quarter (25%), half (50%), full (100%)) gives good results for stain ID and type across samples from the three stain levels. Surprisingly, as summarised in Table 11 above, the application performs better on stains at quarter strength than at half strength.

Next, a library according to the new model was used on washed samples.

TABLE 12
three level model applied to washed stains

| Stain | Stain | | Results on Washed Full Strength | | |
|---|---|---|---|---|---|
| no. | ID | Stain Type | Samples | | |
| 1 | a | Enzyme | | | |
| 2 | b | Fatty | | | |
| 3 | c | Fatty | | | |
| 4 | d | Enzyme | | | |
| 5 | e | Blank | | | |
| 6 | f | Enzyme | o | o | m |
| 7 | g | Bleach | | | |
| 8 | h | Enzyme | s | o | o |
| 9 | i | Fatty | | | |
| 10 | j | Enzyme | t | m | s |
| 11 | k | Particulate | | | |
| 12 | l | Particulate | | | |
| 13 | m | Particulate | m | t | m |
| 14 | n | Enzyme | m | s | s |
| 15 | o | Fatty | | | |
| 16 | p | Fatty | | | |
| 17 | q | Fatty | | | |
| 18 | r | Bleach | blank | s | blank |
| 19 | s | Bleach | s | s | blank |
| 20 | t | Bleach | t | s | blank |
| 21 | u | Fatty | | | |
| 22 | v | Fatty | | | |
| 23 | w | Fatty | | | |
| 24 | x | Fatty | | | |

Correct=5/27 (19%)

Correct type=7/27 (26%)

This model was far less successful and it can therefore be concluded that whilst fresh stains may be identified across three levels (quarter, half and full), washed stains require a different approach.

### Example 6—Wavelength Range Dependence

As for previous examples, NIR spectral data were obtained for the 1-24 known stains (full strength) on cotton. The internal consistency of the resulting library was tested as a function of selected wavelength ranges within these data. Table 13 below shows the success rate for various different NIR wavelength ranges used in the analysis.

TABLE 13
Success rate for different wavelength Ranges

| Range/nm | % Correct Stain | % Correct Stain Type |
|---|---|---|
| 1595-2397 | 79 | 86 |
| 1595-1672 | 45.9 | 65 |
| 1600-2000 | 81.4 | 90 |
| 1800-1877 | 73 | 79 |
| 2000-2397 | 75 | 87 |
| 1800-2200 | 74.5 | 82.5 |

It can be seen that wavelength ranges which span over longer wavelengths give a greater success rate. Although starting at a lowest wavelength value of 1595 or longer was found to be advantageous, the sub-range 1595 nm to 1672 nm was found to be considerably less effective as compared to sub-ranges at longer wavelengths. As can be seen from Table 13 above, a particularly advantageous range of wavelengths was found to be 1600-2000 nm. Scanning over this entire range of wavelengths and comparing the resulting spectra to reference spectra over the entire range gives rise to a significantly higher percentage of identified stain type (90%) over the wide range of stain types tested. This goes against the teaching of the prior art. For example CN 102720034 discloses that separate scans ranging from wavelengths as low as 1400 nm to wavelengths as high as 2526 should be used to correctly identify “food”, “blood” and “mixed” stains. We therefore propose that the improved scanning range of 1600-2000 nm provides good results over an advantageously smaller total scanning range.

The smaller scanning range is particularly advantageous as the NIR scanner itself does not need to function over a large range (thereby simplifying the choice of radiation source). Not only does this have implications for the cost of the NIR scanner required, but it also means that the scanning process itself will be quicker as compared to prior art designs which require a plurality of separate scan ranges in order to identify a plurality of different stains.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

All references referred to above are hereby incorporated by reference.

1. A method for identifying a stain on a textile, the method comprising the steps of:
performing near-infrared reflectance spectroscopy to obtain spectral data from the stain over a range of wavelengths ranging from a shortest wavelength to a longest wavelength, the shortest wavelength having a value of 1595 nm or longer;
accessing reference spectral data of known stains; and
comparing the spectral data of the stain to the reference spectral data of known stains to identify the stain.

2. The method of claim 1, wherein the range of wavelengths over which data is obtained is defined by:
a lower bound corresponding to a shortest wavelength which is no shorter than 1600 nm; and
an upper bound corresponding to a longest wavelength which is no longer than 2000 nm;
wherein the comparison of the spectral data of the stain to the reference spectral data is made over the entire range from 1600 nm to 2000 nm.

3. The method of claim 1, wherein the longest wavelength is no longer than 2397 nm.

4. The method of claim 1, wherein the step of comparing the spectral data of the stain to reference spectral data of known stains includes:
performing multivariate data analysis techniques on the spectral data of the stain to be treated.

5. The method of claim 1, further comprising:
providing a user interface for the user treating the stain to provide information on the stain; and
comparing the information provided at the user interface with information about the known stains.

6. The method of claim 5, wherein the user interface is provided on a mobile device which includes a camera.

7. The stain detection system of claim 5, wherein the user interface includes a typographical interface and/or drop down menu for a user to input information about the stain and/or the textile.

8. A method of treating a textile comprising a stain, the method including the steps of:
identifying a stain on the textile using the method of claim 1;
choosing a treatment regime for treating the textile based on the identified stain.

9. A stain determination system for identifying a stain on a textile including:
a near-infrared reflectance (NIR) spectrometer which operates over a range of wavelengths, the range defined by a lower bound corresponding to a shortest wavelength; and an upper bound corresponding to a longest wavelength, the shortest wavelength being 1595 nm or longer; and
an analysis module comprising a storage means,
the analysis module configured to receive the spectral data from the NIR spectrometer; to retrieve reference spectral data of known stains from the storage means; and to compare the spectral data from the NIR spectrometer with the spectral data of known stains over the entire range of wavelengths, whereby a match between the NIR spectral data and the reference spectral data for a specific stain identifies the stain on the textile.

10. The stain determination system of claim 9, wherein the range of wavelengths over which data is obtained is defined by:
a lower bound corresponding to a shortest wavelength which is no shorter than 1600 nm; and
an upper bound corresponding to a longest wavelength which is no longer than 2000 nm; and
wherein the comparison of the spectral data of the stain to the reference spectral data is made over the entire range from 1600 nm to 2000 nm.

11. The stain determination system of claim 9, further comprising a treatment module, the treatment module configured to select treatment parameters based on the identified stain.

12. The stain determination system of claim 9, wherein the analysis module includes a user interface configured to receive information about the stain from a user.
